(19) United States
(12) Patent Application Publication
(10) Pub. No.: US 2017/0128072 A1
(43) Pub. Date: May 11, 2017

(54) SUTURELESS ANASTOMOSIS DEVICE

(71) Applicant: The Regents of the University of California, Oakland (CA)

(72) Inventors: Aijun Wang, Davis, CA (US); David Boudreault, Davis, CA (US); Nicole Sitkin, Davis, CA (US); Christopher Pivetti, Davis, CA (US)

(21) Appl. No.: 15/318,314

(22) PCT Filed: Jun. 12, 2015
(86) PCT No.: PCT/US15/35587

§ 371 Date: Dec. 12, 2016

Related U.S. Application Data
(60) Provisional application No. 62/012,223, filed on Jun. 13, 2014.

(51) Int. Cl. A61B 17/11; A61B 34/00; A61B 17/00

(52) U.S. Cl. CPC A61B 17/11; A61B 17/00491; A61B 34/73; A61B 2017/00411; A61B 2017/00477; A61B 2017/00876; A61B 2017/1107; A61B 2017/1132

(57) ABSTRACT

A sutureless anastomosis device is provided herein comprising an outer tube defining an outer lumen and an inner tube formed of a compliant material defining an inner lumen. The inner tube is disposed within the outer lumen. In various embodiments, the anastomosis device is implanted such that proximal ends of the outer and inner tubes are secured around an outer surface of a proximal blood vessel, the distal end of the outer tube is secured to an outer surface of a distal blood vessel, and the distal end of the inner tube is secured to an inner surface of the distal blood vessel. Such a configuration connects the proximal blood vessel and distal blood vessel together in a manner that limits or eliminates turbulent flow through the anastomosis device. Methods of implantation are also disclosed.

### FIELD OF THE INVENTION

The present invention relates generally to surgical anastomosis, and specifically, to improved anastomosis grafts and related procedures.

### BACKGROUND

Surgical anastomosis may be performed to connect two tubular structures. A common form of surgical anastomosis is end-to-end anastomosis in which a proximal end of one tubular structure is secured to a distal end of another tubular structure. Surgical anastomosis may be performed, for example, to connect two portions of a blood vessel, ureter, extrahepatic bile duct, esophagus, small intestine or large intestines. In particular, venous and arterial anastomosis procedures are performed as common treatments for vascular diseases, with over 500,000 vascular grafts used in bypass procedures (coronary artery and peripheral vessel) each year. When a portion of a blood vessel becomes badly diseased or damaged, the portion may be surgically excised and replaced with a conduit referred to as an interposition graft. Currently, the interposition graft of choice is autologous vascular tissue from a donor site, such as, for example, the greater saphenous vein. Unfortunately, a variety of problems are associated with this approach. For example, autologous vessel is not always available. Often times, patients requiring such a procedure have systemically diseased or damaged vessels that are not viable grafts. Additionally, even when autologous tissue can be used, special hand-sewn suturing techniques are needed to prevent leakage of blood from the anastomosis sites, and the procedure is tedious and time-consuming. Such a technique is disclosed in Alghoul, M. S., et al., From simple interrupted to complex spiral: A systematic review of various suture techniques for microvascular anastomoses, Microsurgery, 2011, 31(1): 72-80. Moreover, the use of venous and arterial grafts is limited by their 15-35% ten-year failure rate. Such a high failure rate is due to a number of factors including injury to the blood vessel during the procedure, which may lead to thrombus formation following the procedure, as well as arterial inflammation, neointimal hyperplasia, and negative remodeling.

Artificial vascular grafts represent a preferred substitute to native vascular grafts because they can be produced in a large quantity with various lengths and diameters, and they can be made available to patients whose own vessels are not viable graft options. Conventional artificial vascular grafts are hollow conduits that are sutured to native vessels by anastomosis on both ends, requiring the same hand-sewn suturing techniques described above. In many cases, the mismatch of diameters between the vascular grafts and the vessels creates turbulent blood flow, which may lead to neointima formation and subsequent occlusion and failure of the artificial vascular grafts.

Accordingly, a significant need still exists for improved artificial interposition vascular grafts and other anastomosis devices.

### SUMMARY

In particular, there is a significant need for anastomosis devices that can be attached without sutures so as to avoid surgical trauma to adjoining vessels and resultant thrombus formation. There is also a significant need for anastomosis devices that are specially shaped so as to avoid causing turbulent blood flow. Turbulence has been shown to cause neointima formation, which can lead to occlusion and failure of vascular grafts over time. Various aspects disclosed herein may fulfill one or more of these needs.

The systems and methods described herein each have several aspects, no single one of which is solely responsible for its desirable attributes. Without limiting the scope of this disclosure as expressed by the claims that follow, the more prominent features will now be discussed briefly. After considering this discussion, and particularly after reading the section entitled “Detailed Description,” one will understand how the sample features described herein provide for improved anastomosis devices and methods.

One aspect of the present disclosure is directed to a sutureless anastomosis device. In various embodiments, the device comprises an outer tube defining an outer lumen and an inner tube formed of a compliant material defining an inner lumen. The inner tube is disposed within the outer lumen, and the outer and inner tubes each have a proximal end and a distal end, the proximal end being upstream of the distal end, when implanted. The device further comprises a proximal securement mechanism configured to secure the proximal ends of the outer and inner tubes around an outer surface of a proximal blood vessel, and a distal securement mechanism configured to secure the distal end of the outer tube to an outer surface of a distal blood vessel and the distal end of the inner tube to an inner surface of the distal blood vessel.

The proximal securement mechanism may comprise a tube clamp. Additionally or alternatively, the proximal securement mechanism and/or the distal securement mechanism may comprise a plurality of protrusions, a magnet or a plurality of magnets, or a surgical adhesive.

Another aspect of the present disclosure is directed to a sutureless anastomosis device implanted so as to connect two blood vessels together. The device of various embodiments includes an outer tube defining an outer lumen and an inner tube formed of a compliant material defining an inner lumen. The inner tube is disposed within the outer lumen, and the outer and inner tubes each have a proximal end and a distal end. Furthermore, in such embodiments, the proximal ends of the outer and inner tubes are secured around an outer surface of a proximal blood vessel, the distal end of the outer tube is secured to an outer surface of a distal blood vessel, and the distal end of the inner tube is secured to an inner surface of the distal blood vessel so as to connect the proximal blood vessel and distal blood vessel together to allow the flow of blood therethrough.

In at least some embodiments of the devices described above, the inner lumen tapers from the proximal end to the distal end, and the outer and inner tubes share a central axis. The outer tube may be formed of a non-compliant material. The inner tube may include pieces or bands of metal disposed in or around the inner tube, the metal being responsive to magnetic forces. The device may further comprise one or more magnets positioned on an outer surface of the distal end of the outer tube, the magnets applying an attractive force to the inner tube. Additionally or alternatively, the device may further include a plurality of electrical connectors configured to connect to a power source to apply an electric field to the anastomosis device.

An additional aspect of the disclosure is directed to a method of connecting, end to end without sutures, a proximal blood vessel and a downstream distal blood vessel. In various embodiments, the method includes providing a sutureless anastomosis device formed of an outer tube defining an outer lumen and an inner tube made of a compliant material defining an inner lumen, the inner tube disposed within the outer lumen. The method further includes: positioning proximal ends of the inner and outer tubes onto an outer surface of a proximal blood vessel; securely fastening the proximal ends of the outer and inner tubes to the proximal blood vessel; positioning a distal end of the inner tube into a vessel lumen of a distal blood vessel; positioning a distal end of the outer tube around an outer surface of the distal blood vessel; and fastening the distal end of the inner tube to an inner surface of the distal blood vessel, and the distal end of the outer tube to an outer surface of the distal blood vessel.

The method may further include providing a magnetic collar around the distal end of the outer tube, the magnetic collar applying a magnetic force to the distal end of the inner tube to expand and attract the inner tube towards the outer tube so as to securely engage the distal blood vessel between the inner and outer tubes of the anastomosis device.

Additionally or alternatively, the method may further include arresting blood flow through the proximal and distal blood vessels prior to positioning the sutureless anastomosis device, and resuming blood flow following application of a magnetic force on the inner tube, wherein resuming blood flow provides a radially projecting force throughout the inner tube, further securing the distal blood vessel between the inner tube and the outer tube.

Those skilled in the art will appreciate that the foregoing is a summary and thus, contains by necessity, simplifications and omissions of detail. Any particular device or method may have some or all these features or additional or alternative features. Other aspects, features, and advantages will become apparent in the teachings that follow.

### BRIEF DESCRIPTION OF THE DRAWINGS

The above-mentioned aspects, as well as other features, aspects, and advantages of the present technology will now be described in connection with various embodiments, with reference to the accompanying drawings.

FIG. 1A is a schematic representation of a prior art anastomosis device, which sandwiches a vessel between an inner tube and an outer tube on both a proximal end and a distal end of the device. The turbulent flow created from such a device is also illustrated.

FIG. 1B is a schematic representation of a prior art anastomosis device, which is disposed over an outer surface of a vessel on both a proximal end and a distal end of the device. The turbulent flow created from such a device is also illustrated.

FIG. 2A is a schematic representation of one embodiment of an anastomosis device constructed in accordance with the principles of the present invention.

FIG. 2B is a schematic representation of an exemplary approach to implanting the anastomosis device of FIG. 2A.

FIG. 3 depicts a rendering of one embodiment of a securement mechanism constructed in accordance with the principles of the present invention.

FIG. 4 is a schematic representation of another embodiment of the anastomosis device of FIG. 2A.

FIG. 5 is a photograph of a prototype of one embodiment of an anastomosis device constructed in accordance with the principles of the present invention.

FIG. 6 is a block flow diagram showing an example method of sutureless anastomosis, in accordance with the principles of the present invention.

### DETAILED DESCRIPTION

In the following detailed description, reference is made to the accompanying drawings, which form part of the present disclosure. The embodiments described in the drawings and description are intended to be exemplary and not limiting. As used herein, the term “exemplary” means “serving as an example or illustration” and should not necessarily be construed as preferred or advantageous over other embodiments. Other embodiments may be utilized and modifications may be made without departing from the spirit or the scope of the subject matter presented herein. Aspects of the disclosure, as described and illustrated herein, can be arranged, combined, and designed in a variety of different configurations, all of which are explicitly contemplated and form part of this disclosure.

Unless otherwise defined, each technical or scientific term used herein has the same meaning as commonly understood by one of ordinary skill in the art to which this disclosure belongs. In accordance with the claims that follow and the disclosure provided herein, the following terms are defined with the following meanings, unless explicitly stated otherwise.

The term “about,” “approximately,” or “substantially” when used before a numerical designation or range (e.g., pressure or dimensions), indicates approximations which may vary by (+) or (−) 5%, 1% or 0.1%.

As used in the specification and claims, the singular form “a”, “an” and “the” include both singular and plural references unless the context clearly dictates otherwise. For example, the term “a magnet” may include, and is contemplated to include, a plurality of magnets. At times, the claims and disclosure may include terms such as “a plurality,” “one or more,” or “at least one;” however, the absence of such terms is not intended to mean, and should not be interpreted to mean, that a plurality is not conceived.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a device or method consisting essentially of the elements as defined herein would not exclude other materials or steps that do not materially affect the basic and novel characteristic(s) of the claimed invention. “Consisting of” shall mean that the devices, systems, and methods include the recited elements and exclude anything more than a trivial or inconsequential element or step. Embodiments defined by each of these transitional terms are within the scope of this disclosure.

“Anastomosis,” as used herein, shall mean the surgical connection of two tubular structures.

An “anastomosis device” shall refer to an artificial graft, which serves as a conduit and extension connecting two tubular structures. It will be appreciated by those skilled in the art that while anastomosis of blood vessels, particularly veins and arteries, is primarily discussed herein, each of the provided devices may alternatively be used to facilitate the connection of two portions of a ureter, extrahepatic bile duct, esophagus, small intestine, large intestines, or any other tubular structure.

As used herein, “sutureless” shall mean connecting without the use of sutures, stitches, staples, or other manual, seam-forming connectors.

As used herein, “distal” and “proximal” are relational terms, wherein “proximal” refers to a vessel, a portion of a vessel, or a portion of a medical device that is upstream relative to a downstream “distal” vessel, portion of the vessel, or portion of the medical device. For example, the proximal end of an anastomosis device is the end which, when implanted within a body, is positioned upstream relative to the distal end of the anastomosis device.

“Upstream” and “downstream” are also relative terms referring to the direction of blood flow in a vessel. Blood flows from the upstream location to the downstream location.

As used herein, “compliant” shall refer to materials that are flexible, deformable, and capable of at least some expansion and compression.

As used herein, “securement mechanism” refers to components or features provided to secure one or more ends of the anastomosis device to a vessel wall. Suitable securement mechanisms may include, for example, any of a variety of clamps, which apply pressure on an end of the anastomosis device to tighten the end around a vessel wall. As described more below, the securement mechanism may additionally or alternatively include a magnetic collar, barbs or other protrusions for engagement, adhesives, elastic materials, or any other acceptable devices, which can be implanted into a body to secure the anastomosis device to the vessel walls. In some embodiments, the securement mechanism comprises a snap fit or a friction fit between the vessel wall and the anastomosis device.

As used herein, “magnet” is a material or object that produces a magnetic field, said magnetic field producing an attractive force on magnetic-responsive materials and a force that attracts or repels other magnets. “Magnetic-responsive” materials may include ferromagnetic and paramagnetic materials, for example.

As used herein, “protrusions for engagement” may include barbs, spikes, or any other features that extend from a surface and are sharp so as to cause the extended features to at least partially pierce the wall of a vessel upon contacting the vessel.

Various embodiments disclosed herein are directed to anastomosis devices designed to: avoid stricture and/or tension at the site of anastomoses, preserve blood flow without producing severe narrowing or extra turbulence, provide a faster method to create an anastomosis, and/or reduce graft failure caused by intimal hyperplasia and restenosis. Such characteristics provide significant improvements over known anastomosis devices.

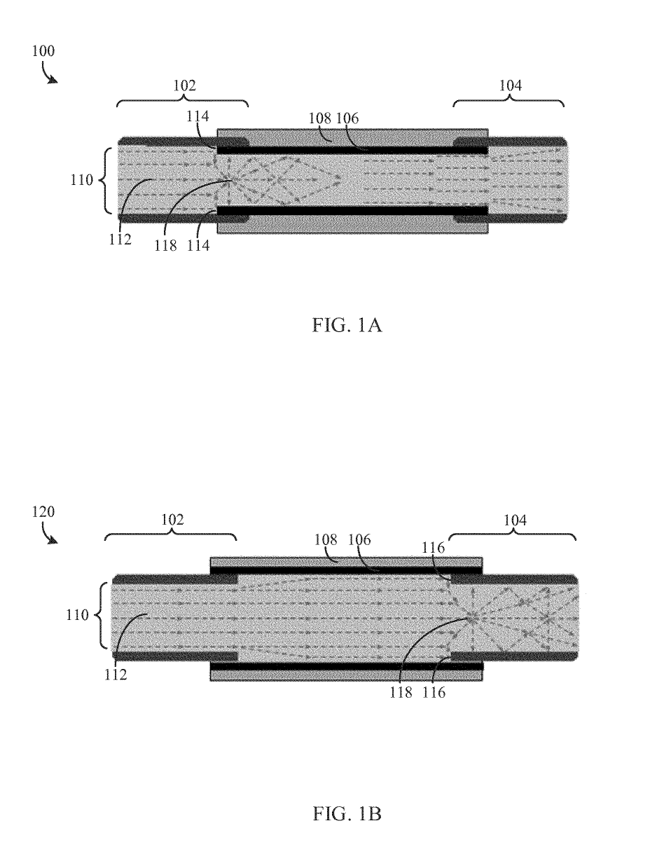

Referring to FIGS. 1A and 1B, others have conceived of sutureless artificial grafts for use in anastomosis; however, to date, none have overcome the plurality of problems noted above. Examples of such grafts include U.S. Appl. Pub. No. 2004/0249399 to Cinquin et al. and U.S. Pat. No. 5,916,266 to Tozzi, the disclosures of which, are herein incorporated by reference in their entireties. As shown in FIG. 1A and FIG. 1B, previously proposed artificial anastomosis devices generate a significant turbulent flow 118 within an inner tube 106 of a device or an attached distal blood vessel 104. For example, referring to FIG. 1A, a first anastomosis device 100 according to the prior art is formed of an inner tube 106 concentrically disposed within an outer tube 108. The inner tube 106 is disposed within a blood vessel lumen 110 defined by a proximal blood vessel 102 and a distal blood vessel 104. The outer tube 108 is disposed on an outer surface of both blood vessels.

As another example, referring to FIG. 1B, a second anastomosis device 120 according to the prior art positions both the inner tube 106 and the outer tube 108 on an outer wall of both blood vessels. The first device 100 and the second device 120 each cause a structural component such as a device edge 114 (i.e., an upstream edge of the inner tube 106 facing an incoming blood flow 112 from the proximal vessel 102, as shown in FIG. 1A) or a vessel edge 116 (i.e., an upstream edge of the distal vessel 104 facing an incoming blood flow 112 from the inner tube 106, as shown in FIG. 1B) to protrude into the lumen 110 and into the blood flow 112. Such structural components act effectively as a partial barrier to blood flowing downstream. When the blood flow 112 hits the structural component (e.g., the device edge 114 or the vessel edge 116), the blood flow 112 path is disrupted and its direction modified, thereby resulting in a turbulent flow 118. The resultant non-uniform turbulent flow 118 causes blood cells to migrate and aggregate at various locations in the inner tube 106 or the distal vessel 104, causing neointima formation. Such formation can lead to occlusion and failure of vascular grafts over time.

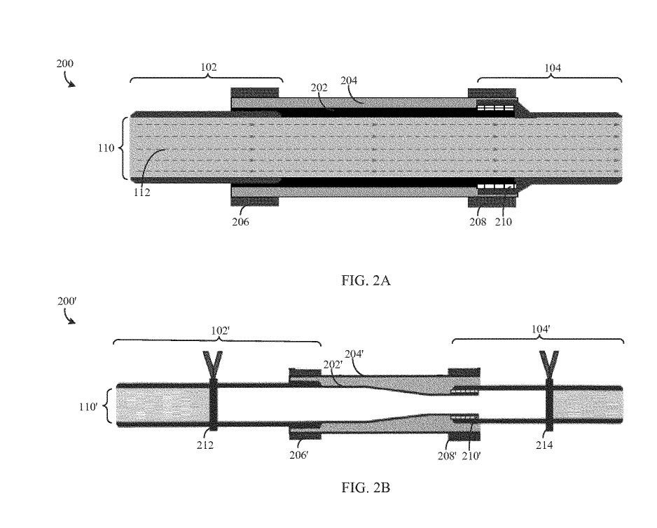

Referring to FIG. 2A, an improved anastomosis device 200 is configured to avoid flow-disrupting projections into the luminal space. As shown by the arrows representing the blood flow 112, in the device 200, a laminar or near-laminar blood flow 112 is maintained.

In various embodiments, the anastomosis device 200 of the present disclosure is bilaminar. That is, it is formed of two tubes, one disposed within the other. Additionally, the device 200 is attachable to a vessel wall without the need for sutures.

The anastomosis device 200 includes an inner tube 202 concentrically disposed within an outer tube 204, a proximal collar 206, and a distal collar 208. In various embodiments, the outer tube 204 is non-compliant (i.e., non-deformable, non-compressible, non-expandable, and/or rigid). The outer tube 204 may be cylindrically shaped. The outer tube 204 can be formed in a variety of lengths and diameters to match the dimension requirements of an anastomosis of the proximal vessel 102 to the distal vessel 104. For example, the outer tube 204 may have a diameter slightly larger than the portions of the proximal vessel 102 and the distal vessel 104 to which it is connecting. In some embodiments, the distal end of the outer tube 204 has a diameter that is just slightly larger than the diameter of the distal vessel 104. The diameter at a proximal end of the outer tube 204 may be equal to or slightly larger than the diameter at a distal end of the outer tube 204. The length of the inner tube 202 and the outer tube 204 may be selected to be larger than the length of a gap between the proximal vessel 102 and the distal vessel 104, thereby allowing for overlap between the device 200 and the vessel portions. Such overlap can provide secure connections between the device 200 and the vessel portions. In some embodiments, the device 200 is provided in a plurality of sizes, such as, for example, small, medium, and large, where the size refers to the diameter of the outer tube 204. In some embodiments, the device 200 can be cut to a desired length.

The inner tube 202 is positioned within the lumen of the outer tube 204 and shares an elongated central axis with the outer tube 204. The inner tube 202 may be compliant and funnel-shaped when not actively engaged to blood vessels. In various embodiments, the inner tube 202 is formed of a flexible polymer and has a diameter which tapers from a larger proximal end to a smaller distal end (e.g., as shown in FIG. 2B). The diameter of the proximal end of the inner tube 202 is sized to fit around a portion of the proximal vessel 102 and the diameter of the distal end is sized to fit within a portion of the distal vessel 104.

In use, both the inner tube 202 and the outer tube 204 attach to the external surface of the proximal vessel 102. The inner tube 202 may be in direct contact with an outer surface of the proximal vessel 102 and the outer tube 204 is in contact with the inner tube 202.

The proximal end of the device 200 is configured to be tightly secured to the proximal vessel 102 without narrowing the lumen 110. In some embodiments, the proximal end of the device 200 is secured to the proximal vessel 102 with the proximal collar 206. The proximal collar 206 is positioned around a portion of the outer surface of the outer tube 204 that is annularly disposed about a downstream end of the proximal vessel 102. The proximal collar 206 may be adjustable in size so that it can be tightened in place around the outer tube 204. Alternatively, the proximal collar 206 may be specifically sized to have a circumference fitted for the proximal vessel 102. A properly sized or properly tightened proximal collar 206 will apply a force onto the device 200 and the portion of the proximal vessel 102 disposed therein, which is: (1) sufficient to create a friction fit that holds the device 200 in place against the proximal vessel 102, and (2) insufficient to cause significant narrowing of the portion of the lumen 110 defined by the proximal vessel 102. In some embodiments, the proximal collar 206 is a tube clamp.

A distal end of the anastomosis device 200 is configured to sandwich the distal vessel 104 between the inner tube 202 and the outer tube 204. In use, the narrower inner tube 202 is positionable within the lumen 110 at the distal vessel 104 and the outer tube 204 is positionable around an outer surface of the distal vessel 104. In some embodiments, the device 200 uses magnetic force to securely hold the distal vessel 104 between the inner tube 202 and the outer tube 204. In such embodiments, at least a distal end of the inner tube 202 contains a material that will respond to a magnet 210. In some such embodiments, the material is a flexible magnetic-responsive collar or sheath disposed around an outer surface of the distal end of the inner tube 202. For example, a metallic ring may be embedded into a synthetic polymer material of the inner tube 202 through known bioengineering molding processes in order to form an integral structure. In other embodiments, a plurality of magnets 210 may be spaced around the outer surface of the distal end of the inner tube 202 and affixed to the outer surface of the inner tube 202 during, for example, the molding process. In still other embodiments, at least a portion of the distal end of the inner tube 202 comprises a magnetic-responsive material. For example, pieces, flakes, or shavings of a magnetic-responsive material may be dispersed within the polymer material of the inner tube 202, or thin, magnetic-responsive filaments may be woven among the polymer material of the inner tube 202. In yet another embodiment, the inner tube 202 includes magnetic-responsive material that extend the length of the inner tube 202, allowing the inner tube 202 to be measured and cut to fit individual applications (e.g., a defect length of a blood vessel) while maintaining magnetic-responsive properties.

Advantageously, by allowing the proximal vessel 102 to be connected to both the inner tube 202 and the outer tube 204 on the outside of the wall of the proximal vessel 102, the endothelium of the proximal vessel 102 is left intact and the blood flow 112 can proceed antegrade without interruption or turbulence. By keeping the endothelium intact, blood vessels are subjected to less injury and endothelium may grow and become integrated into the inner tube 202 of the device 200, thereby promoting healing and longevity of the device 200. Additionally, the two-layer construct with a compliant inner tube 202 and a non-complaint outer tube 204 offers the advantage of maintaining a rigid outer layer, which prevents against excessive dilation and eventual pseudo-aneurysm. At the same time, the inner tube 202 is configured to expand to the limits set by the outer tube 204 much like an arterial wall expands and contracts depending on the pressure exerted. By mimicking the compliance of a blood vessel wall, more fluid and natural blood flow can be achieved, which may produce a more natural healing response.

Referring to FIG. 2B, the device 200′ is constructed similar to the device 200 of FIG. 2A, wherein like components are identified by like-primed reference numbers. For example, the inner tube 202′ of FIG. 2B corresponds to the inner tube 202 of FIG. 2A, the outer tube 204′ of FIG. 2B corresponds to the outer tube 204 of FIG. 2A, and so on. However as shown in FIG. 2B, anastomosis of the proximal vessel 102′ and the distal vessel 104′ using the device 200′ may be accomplished through the use of clamps. For example, a proximal clamp 212 (e.g., a vascular clamp) may be engaged to the proximal vessel 102′ at an upstream location relative to a damaged vessel segment, and a distal clamp 214 may be engaged to the distal vessel 104′ at a downstream location relative to the damaged vessel segment. The engagement of the proximal clamp 212 and the distal clamp 214 prevents blood (e.g., as would otherwise be provided by the blood flow 112 shown in FIG. 2A) from entering the lumen 110′ between the two clamps.

Upon preventing the blood flow 112 from reaching the space between the proximal vessel 102′ and the distal vessel 104′, the device 200′ may be installed in any of several ways. In one embodiment, the proximal end of the device 200′ (i.e., including both the inner tube 202′ and the outer tube 204′) first annularly engages the outer surface of the downstream end of the proximal vessel 102′, and is secured by the proximal collar 206′. In turn, the upstream end of the distal vessel 104′ is inserted into the annular lumen between the inner tube 202′ and the outer tube 204′ at the distal end of the device 200′. The distal collar 208′ secures the device 200′ to the upstream end of the distal vessel 104′, and one or more of the magnets 210′ disposed in the inner tube 202′ causes the diameter of the inner tube 202′ to expand, thereby engaging the inner circumference of the outer tube 204′. Upon securing the device 200′ to both the proximal vessel 102′ and the distal vessel 104′, the proximal clamp 212 and the distal clamp 214 may be disengaged, allowing the blood flow to resume.

The device 200′ can also be installed with the use of surgical glue. Surgical glue is a biocompatible adhesive configured to maintain an adhesive property while disposed within a body of a patient. In one such an arrangement, surgical glue is disposed on the inner tube 202′ about a portion of an inner circumference at a proximal end and about a portion of an outer circumference at a distal end. As such, while the blood flow 112 is arrested, the proximal end of the device 200′ is first annularly positioned about the outer circumference of the downstream end of the proximal vessel 102′, such that surgical glue at the proximal end of the inner tube 202′ contacts and adheres to the outer surface of the proximal vessel 102′. The upstream end of the distal vessel 104′ is then positioned between the inner tube 202′ and the outer tube 204′ at the distal end of the device 200′, such that surgical glue contacts and adheres to the inner surface of the distal vessel 104′. The proximal collar 206′ and the distal collar 208′ then engage respective ends of the device 200′, securing the device 200′ to the blood vessels. The proximal clamp 212 and the distal clamp 214 are then removed, restoring the blood flow 112 through the blood vessels.

In another embodiment incorporating the use of surgical glue, surgical glue is applied to an inner circumference of both an inner proximal collar and an inner distal collar. The inner proximal collar and the inner distal collar are rigid or semi-rigid ring-like structures. Upon arresting the blood flow 112, the inner proximal collar is first positioned and engaged to an outer circumference of the proximal vessel 102′ (i.e., via the surgical glue disposed on the inner circumference of the inner proximal collar) and the inner distal collar is positioned and engaged to an outer circumference of the distal vessel 104′. An expandable balloon (e.g., a retrievable elastic capsule that can be deformably expanded and collapsed, for example using a fluid pump or activating/deactivating a shape memory material disposed within) in a collapsed configuration is threaded into the proximal vessel 102′ and subsequently expanded to press the wall of the proximal vessel 102′ circumferentially against the inner proximal collar. The inner tube 202′ with surgical glue disposed about an outer circumference of its distal end is positioned within the upstream end of the distal vessel 104′ such that the distal vessel 104′ is sandwiched between the inner tube 202′ and the inner distal collar. The expandable surgical balloon is threaded through the inner tube 202′ and toward the distal vessel 104′, and is inflated upon reaching the distal end of the inner tube 202′. Inflating the surgical balloon presses the inner tube 202′, the distal vessel 104′, and the inner distal collar together. The surgical balloon is then collapsed and retrieved, after which the proximal end of the inner tube 202′ is positioned about the outer circumference of the inner proximal collar.

Still referring to the same example embodiment incorporating surgical glue, the outer tube 204′ is in the form of a semi-rigid tubular sleeve having a longitudinal slit from its proximal end to its distal end. The inner proximal collar, the inner tube 202′, and the inner distal collar are each inserted into the slit of the outer tube 204′, concentrically disposing those components within the outer tube 204′. An outer proximal collar is annularly engaged to the outer circumference of the outer tube 204′ at its proximal end, thereby sandwiching a proximal portion of the outer tube 204′ between the inner proximal collar and the outer proximal collar. An outer distal collar is correspondingly engaged to the outer circumference of the outer tube 204′ at its distal end. The proximal clamp 212 and the distal clamp 214 may then be removed to restore the blood flow 112.

In addition to or instead of magnets and adhesives, the device 200′ may also be installed using an elastic substructure. For example, in one embodiment, a mesh or weave of elastic material (e.g., biocompatible metals or plastics having elastic properties) can be integrated into all or part of the pliable inner tube 202′. The elastic material is integrated into the inner tube 202′ such that the inner tube 202′ is naturally (i.e., in the absence of an applied external force) in an expanded configuration having a larger diameter. An external compressive force can deform the elastic material to reduce the diameter of the inner tube 202′ to a compressed configuration having a corresponding smaller diameter. However, in the compressed configuration, inherent properties of the elastic material will cause the inner tube 202′ to seek to return to the expanded configuration. In some such embodiments, a ripcord is disposed through or associated with the mesh or weave of the elastic material while the inner tube 202′ is in the compressed configuration, thereby retaining the compressed configuration upon the removal of the external compressive force.

In an embodiment incorporating the elastic material and the ripcord in the inner tube 202′, the elastic material is integrated into a distal portion of the inner tube 202′. The ripcord is disposed about the terminal circumference at the distal end of the inner tube 202′, and maintains the distal portion of the inner tube 202′ in the compressed configuration. The ripcord is associated with a drawstring protruding from the distal end of the inner tube 202′, such that engaging and pulling the drawstring removes the ripcord from the inner tube 202′. The proximal end of the inner tube 202′ and the outer tube 204′ is first positioned about the external circumference of the downstream end of the proximal vessel 102′ (e.g., via any of the approaches discussed above and below). The upstream end of the distal vessel 104′ is positioned between the inner tube 202′ and the outer tube 204′ at the distal end of the device 200′. At this point, a needle deployed through the distal vessel 104′ engages the drawstring and withdraws the drawstring and associated ripcord from the inner tube 202′. Upon withdrawing the ripcord, the elastic material causes the distal portion of the inner tube 202′ to expand into the expanded configuration. The expanding force applied by the elastic material sandwiches the distal vessel 104′ between the inner tube 202′ and the outer tube 204′. The proximal collar 206′ and the distal collar 208′ may then engage and secure the device 200′ to the blood vessels, and the blood flow 112 can be restored.

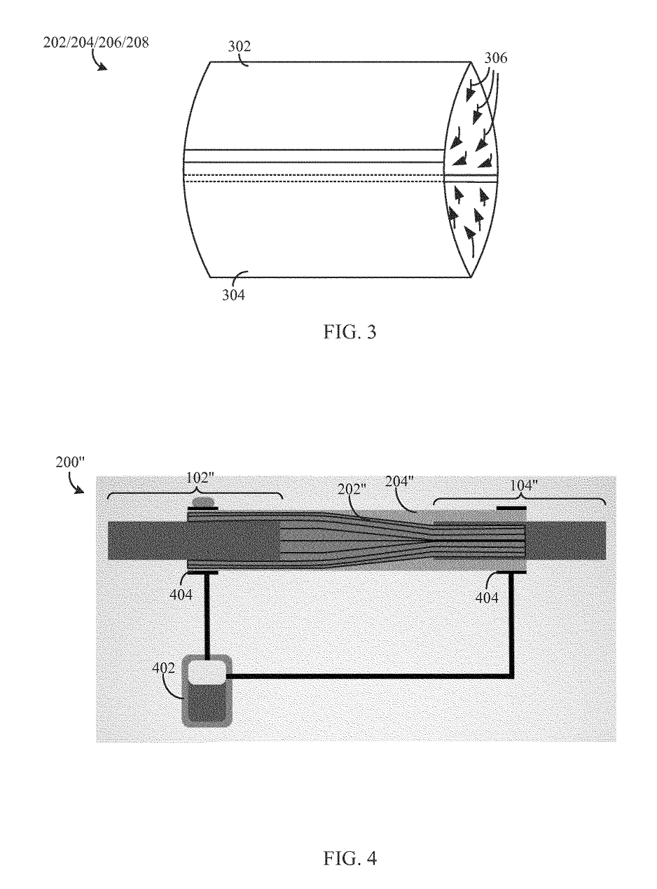

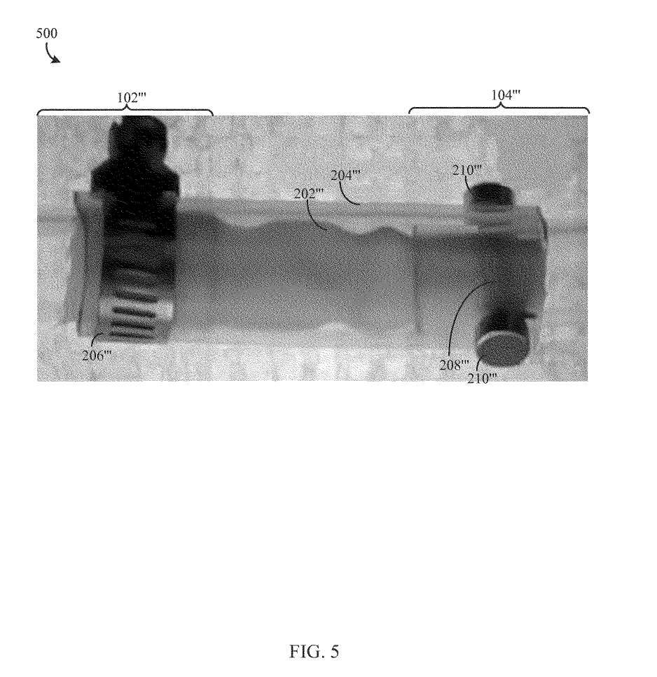

Referring to FIG. 3, in some embodiments, engagement of the device to one or both of the proximal vessel 102 and the distal vessel 104 can be facilitated by a plurality of protrusions 306. The plurality of protrusions 306 may be integrated into the device 200 and configured to pierce or grasp the wall of a vessel portion (e.g., via a plurality of barbs) to further secure the device 200. In some embodiments, the plurality of protrusions 306 are integrated into an interior surface of a first conduit half 302 and a second conduit half 304. The first conduit half 302 and the second conduit half 304 together form respective halves of a conduit. In some embodiments, each half can removably engage the other (e.g., by one or more fasteners, or by having physical features configured to removably snap into each other). In other embodiments, each half is separately formed and later permanently engaged to the other (e.g., glued). In addition, according to various embodiments, the conduit comprising the plurality of protrusions 306 may be disposed on or integrated with an inner tube 202, an outer tube 204, a proximal collar 206, or a distal collar 208.

In one embodiment, an inner surface of the inner tube 202 includes the plurality of protrusions 306 on a proximal end, and upon engagement, further secures the inner tube 202 to the proximal vessel 102 (e.g., by piercing the proximal vessel 102 at a corresponding plurality of points).

In another embodiment, the plurality of protrusions 306 are provided within an inner surface of the proximal collar 206. The outer surface of such the proximal collar 206 may be molded, adhered, or otherwise secured to an inner surface of the proximal end of the inner tube 202. The collar may be formed of a metal, hard plastic, or composite. In some such embodiments, during implantation, the proximal portion of the inner tube 202 and the proximal collar 206 with the plurality of protrusions 306 slips over the proximal vessel 102 while the blood flow 112 is blocked, for example, via the proximal clamp 212 and the distal clamp 214. When the blood flow 112 is resumed, the pressure of the blood causes the proximal vessel 102 to expand slightly, the expansion causing the plurality of protrusions 306 surrounding the proximal vessel 102 to more strongly contact and pierce the wall of the proximal vessel 102.

In another embodiment, a thin membrane may cover the plurality of protrusions 306 during initial insertion of the proximal vessel 102 into the inner lumen of the device 200. Once the device 200 is in place, the membrane may be slid away, causing the plurality of protrusions 306 to deploy and engage the wall of the proximal vessel 102.

In various embodiments in which the plurality of protrusions 306 are provided on a distal end of the device 200, the plurality of protrusions 306 may be integrated into an inner surface of the outer tube 204 or provided within an inner surface of the distal collar 208. In some such arrangements, the distal collar 208 is affixed to the inner surface of the outer tube 204. As such, the plurality of protrusions 306 can be configured to engage and further secure the device 200 to the distal vessel 104 (e.g., with protrusions extending from the outer tube 204 or the distal collar 208 and into the distal vessel 104).

In still other embodiments, the plurality of protrusions 306 may be disposed on an outer wall of the inner tube 202. A metallic stent may be placed inside the distal end of the inner tube 202. Such a stent expands when subjected to a magnetic force applied on the outer surface of the outer tube 204. In one embodiment, the stent expansion causes the inner tube 202 to expand, which causes the plurality of protrusions 306 disposed along the outer surface of the inner tube 202 to engage with the distal vessel 104. The stent may then be removed with the inner tube 202 remaining in an expanded state due to the engagement of the plurality of protrusions 306 with the wall of the distal vessel 104 and further from force generated when the blood flow 112 resumes. As one of skill in the art would appreciate, other arrangements and combinations of the plurality of protrusions 306, magnets, adhesives, and elastic materials to secure an engagement of the device 200 to blood vessel walls are possible.

Referring now to FIG. 4, the device 200″ is constructed similar to the device 200 of FIG. 2A and the device 200″ of FIG. 2B, wherein corresponding components are identified by double-primed reference numbers. In the depicted embodiment, the device 200″ further includes electrically conductive connectors 404 that are configured to be connected to a power source 402. In some arrangements, one of the connectors 404 is engaged to the proximal vessel 102″, and another one of the connectors 404 is engaged to the distal vessel 104″. Connection to the power source 402 causes an electric field to be applied at each of the connectors 404, which may facilitate growth of endothelial cells and tissue within the inner tube 202″ and promote healing. Additionally or alternatively, the device 200″ may be embedded with a slow-release substrate, which is delivered at the point of anastomosis to help promote healing.

Referring now to FIG. 5, a prototype 500 is constructed similar to the device 200 of FIG. 2A, the device 200″ of FIG. 2B, and the device 200′″ of FIG. 4, wherein corresponding components are identified here by triple-primed reference numbers. The prototype 500 shows a pliable inner tube 202′″ concentrically disposed within a rigid outer tube 204′″. The prototype 500 is shown with a proximal end of both the inner tube 202′″ and the outer tube 204′″ engaged to an outer surface of the proximal vessel 102′″. In addition, the proximal collar 206′″ is shown as a tube clamp securing the engagement of the proximal end of the prototype 500 to the proximal vessel 102′″. The distal end of the inner tube 202′″ is shown as disposed within an interior of the distal vessel 104′″, and the distal end of the outer tube 204′″ is shown as annularly disposed about an exterior surface of the distal vessel 104′″. As such, the distal vessel 104′″ is sandwiched between the inner tube 202′″ and the outer tube 204′″. In addition, two magnets 210′″ are disposed about the exterior of the outer tube 204′″ at the distal end of the prototype 500. The magnets 210′″ are shown as engaging the distal collar 208′″, which here, includes a magnetic-responsive material. As such, the magnets 210′″ cause the inner tube 202′″ to expand and engage an inner surface of the distal vessel 104′″.

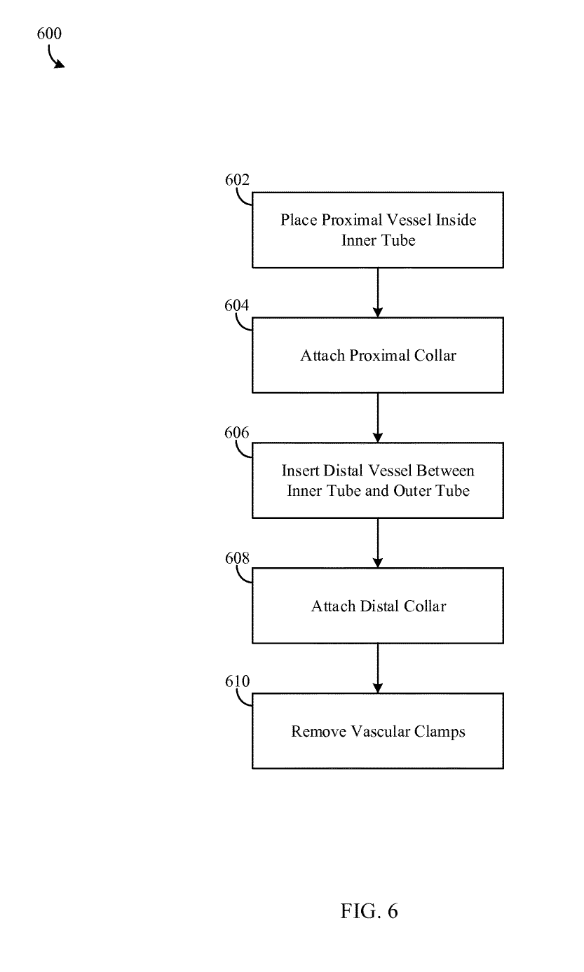

Referring to FIG. 6, an exemplary method 600 of using the device 200 is shown. At 602, a device (e.g., the device 200) is inserted into an injured portion of a body of a patient where a section of a vessel (e.g., a blood vessel) has been removed. In a preferred embodiment, the vessel is an artery. In other embodiments, the vessel is a vein. To facilitate insertion of the device, vascular clamps (e.g., the proximal clamp 212 and the distal clamp 214) are used to arrest blood flow (e.g., the blood flow 112) in the target injured vessel. A proximal end of the anastomosis device is then inserted onto a proximal portion of the target vessel (e.g., the proximal vessel 102) with the proximal ends of both an inner tube (e.g., the inner tube 202) and an outer tube (e.g., the outer tube 204) positioned around an outer surface of the vessel.

At 604, a proximal collar (e.g., the proximal collar 206) is applied over the proximal end of the device to secure the device to the proximal vessel portion. In some embodiments, applying the proximal collar includes positioning and tightening the collar over the proximal end of the anastomosis device.

At 606, a distal end of the anastomosis device is connected to a distal portion of the target vessel (e.g., the distal vessel 104) with a distal end of the inner tube positioned within the lumen (e.g., the lumen 110) of the distal vessel portion and a distal end of the outer tube positioned around the outer surface of the distal vessel portion. In other embodiments, the distal end of the anastomosis device is attached first followed by attachment of the proximal end to the proximal portion of the target vessel. In some embodiments, the distal end of the inner tube of the device includes magnetic-responsive material embedded therein.

At 608, a magnetic distal collar (e.g., the distal collar 208) is secured around the outer surface of the distal end of the outer tube. The magnetic distal collar generates a magnetic field, which exerts an attractive magnetic force on the magnetic-responsive material embedded within the inner tube. The attractive magnetic force pulls the magnetic-responsive material and surrounding material of the inner tube radially outward towards the magnetic distal collar, thereby causing the lumen of the distal end of the inner tube to expand. Such movement causes the distal vessel portion to become sandwiched between the inner tube and the outer tube of the anastomosis device. In various embodiments, securement of the anastomosis device to the distal vessel portion and the proximal vessel portion are both achieved without causing significant injury to the vessel portions.

At 610, the vascular clamps are removed. After the vascular clamps are removed, blood flow resumes through the blood vessels and the device, and the longitudinal flow of blood through the enclosed space of the inner tube generates a force directed radially outward, which can cause further expansion of the distal end of the inner tube. In various embodiments of the method 600, a smooth transition is maintained from the proximal vessel portion to the distal vessel portion with no edges protruding into the luminal space.

In some embodiments, since the endothelium cells of the proximal vessel portion are not damaged during the implantation procedure or from subsequent turbulence, endothelium is present for growth. Over time, endothelium may grow over the inner surface of the inner tube. Such a result may facilitate long-term success of the implanted device.

Throughout and within this specification various technical publications are referenced to more fully describe the state of the art. The disclosures of these references are incorporated herein by reference in their entireties.

Although the foregoing has included detailed descriptions of some embodiments by way of illustration and example, it will be readily apparent to those of ordinary skill in the art in light of the teachings of these embodiments that numerous changes and modifications may be made without departing from the spirit or scope of the appended claims.

What is claimed is:

1. A sutureless anastomosis device, comprising:
an outer tube defining an outer lumen;
an inner tube formed of a compliant material defining an inner lumen, the inner tube disposed within the outer lumen, wherein the outer tube and the inner tube each have a proximal end and a distal end;
a proximal securement mechanism configured to secure the proximal end of the outer tube and the proximal end of the inner tube around an outer surface of a proximal blood vessel; and
a distal securement mechanism configured to secure the distal end of the outer tube to an outer surface of a distal blood vessel and the distal end of the inner tube to an inner surface of the distal blood vessel.

2. The sutureless anastomosis device of claim 1, wherein the proximal securement mechanism comprises a tube clamp.

3. The sutureless anastomosis device of claim 1, wherein at least one of the proximal securement mechanism and the distal securement mechanism comprises a plurality of protrusions for engagement with a corresponding one of the proximal blood vessel and the distal blood vessel.

4. The sutureless anastomosis device of claim 3,
wherein the at least one of the proximal securement mechanism and the distal securement mechanism further comprises a removable membrane selectively in one of a first configuration and a second configuration,
wherein in the first configuration, the removable membrane prevents an engagement of the plurality of protrusions with the outer surface of the corresponding one of the proximal blood vessel and the distal blood vessel, and
wherein in the second configuration, the removable membrane allows an engagement of the plurality of protrusions with the outer surface of the corresponding one of the proximal blood vessel and the distal blood vessel.

5. The sutureless anastomosis device of claim 3, wherein a blood flow within the inner tube causes an engagement of the plurality of protrusions with at least one of the proximal blood vessel and the distal blood vessel.

6. The sutureless anastomosis device of claim 3,
wherein the distal securement mechanism comprises one of a magnet and a magnetically-responsive material, and a distal end of the inner tube comprises the other of a magnet and a magnetically-responsive material; and
wherein a magnetic engagement of the magnet and the magnetically-responsive material engages the plurality of protrusions to the distal blood vessel.

7. The sutureless anastomosis device of claim 1,
wherein the distal securement mechanism comprises one of a magnet and a magnetically-responsive material, and a distal end of the inner tube comprises the other of a magnet and a magnetically-responsive material, and
wherein a magnetic engagement of the magnet and the magnetically-responsive material engageably sandwiches the distal blood vessel between the distal end of the inner tube and the distal end of the outer tube.

8. The sutureless anastomosis device of claim 1, wherein at least one of the proximal securement mechanism and the distal securement mechanism comprises a surgical glue.

9. A sutureless anastomosis device, comprising:
an outer tube defining an outer lumen; and
an inner tube formed of a compliant material defining an inner lumen, the inner tube disposed within the outer lumen, wherein the outer tube and the inner tube each have a proximal end and a distal end;
wherein the proximal end of the outer tube and the proximal end of the inner tube are secured around an outer surface of a proximal blood vessel, the distal end of the outer tube is secured to an outer surface of a distal blood vessel, and the distal end of the inner tube is secured to an inner surface of the distal blood vessel so as to connect the proximal blood vessel and distal blood vessel together to allow the flow of blood therethrough.

10. The sutureless anastomosis device of claim 9, wherein the inner lumen tapers from the proximal end to the distal end of the inner tube.

11. The sutureless anastomosis device of claim 9, wherein the outer tube and the inner tube share a central axis.

12. The sutureless anastomosis device of claim 9, wherein the outer tube is formed of a non-compliant material.

13. The sutureless anastomosis device of claim 9, wherein the inner tube comprises at least one of magnetic-responsive pieces of metal, magnetic-responsive bands of metal, and a magnet.

14. The sutureless anastomosis device of claim 9, further comprising a plurality of electrical connectors configured to connect to a power source to apply an electric field to the anastomosis device.

15. A method of connecting, end to end without sutures, a proximal blood vessel and a downstream distal blood vessel, the method comprising:
providing a sutureless anastomosis device comprising: an outer tube defining an outer lumen, and an inner tube formed of a compliant material defining an inner lumen, the inner tube disposed within the outer lumen, wherein the outer tube and the inner tube each have a proximal end and a distal end;
positioning the proximal end of the inner tube and the proximal end of the outer tube onto an outer surface of the proximal blood vessel;
fastening the proximal end of the outer tube and the proximal end of the inner tube to the proximal blood vessel;
positioning the distal end of the inner tube into a vessel lumen of the distal blood vessel and the distal end of the outer tube around an outer surface of the distal blood vessel; and
fastening the distal end of the inner tube to an inner surface of the distal blood vessel, and the distal end of the outer tube to an outer surface of the distal blood vessel.

16. The method of claim 15, further comprising providing a magnetic collar around the distal end of the outer tube, wherein the magnetic collar fastens the distal end of the inner tube and the distal end of the outer tube to the distal blood vessel by applying a magnetic force to the distal end of the inner tube to expand and attract the inner tube towards the outer tube so as to securely engage the distal blood vessel between the inner tube and the outer tube.

17. The method of claim 16, further comprising arresting a blood flow through the proximal blood vessel and the distal blood vessel prior to positioning the sutureless anastomosis device; and resuming the blood flow following application of the magnetic force on the inner tube, wherein resuming the blood flow provides a radially projecting force throughout the inner tube, further securing the fastening of the distal blood vessel between the inner tube and the outer tube.

18. The method of claim 15, further comprising engaging a connector in electrical communication with a power source to at least one of the proximal blood vessel and the distal blood vessel; and generating, by the power source, an electrical field at the connector.

19. The method of claim 15, wherein the distal end of at least one of the inner tube and the outer tube comprises a plurality of protrusions, and wherein fastening includes positioning a stent within the distal end of the inner tube and radially expanding the stent within the inner tube sufficient to cause an engagement of the plurality of protrusions to the distal blood vessel.

20. The method of claim 15, wherein the distal end of the inner tube comprises an elastic material in a compressed configuration during the positioning of the distal end of the inner tube, and wherein fastening the distal end of the inner tube to the inner surface of the distal blood vessel includes disposing the elastic material in an expanded configuration.
